(19) United States
(12) Patent Application Publication
(10) Pub. No.: US 2026/0035624 A1
(43) Pub. Date: Feb. 5, 2026

(54) RECYCLED CONTENT PARAXYLENE AND RELATED CHEMICAL COMPOUNDS FROM WASTE PLASTIC

(71) Applicant: Eastman Chemical Company, Kingsport, TN (US)

(72) Inventors: Nick Allen Collins, Fall Branch, TN (US); Daryl Bitting, Longview, TX (US); Michael Gary Polasek, Richmond, TX (US); Xianchun Wu, Longview, TX (US); David Eugene Slivensky, Tatum, TX (US)

(73) Assignee: Eastman Chemical Company, Kingsport, TN (US)

(21) Appl. No.: 19/099,831

(22) PCT Filed: Jul. 20, 2023
(86) PCT No.: PCT/US2023/070571

§ 371 Date: Jan. 30, 2025

Related U.S. Application Data
(60) Provisional application No. 63/370,247, filed on Aug. 3, 2022.

(51) Int. Cl. C10G 55/04; C07C 51/255; C07C 51/36; C07C 63/26

(52) U.S. Cl. CPC C10G 55/04; C07C 51/255; C07C 51/36; C07C 63/26; C10G 2300/1003; C10G 2400/30

(57) ABSTRACT

Processes and facilities for producing a recycled content organic chemical compound directly or indirectly from waste plastic. Processing schemes are described herein for converting waste plastic (or hydrocarbon having recycled content derived from waste plastic) into useful intermediate chemicals and final products. In some aspects, recycled content aromatics (r-aromatics) can be processed to provide recycled content paraxy lene (r-paraxylene), which can then be used to provide recycled content terephthalic acid (r-TPA) and/or recycled content polyethylene terephthalate (r-PET). In some aspects, the r-PET can include one or more comonomers to form r-co-PET, which can be used in a variety of end use applications.

### BACKGROUND

Aromatic compounds such as benzene, toluene, and xylenes are important industrial chemicals used in a variety of applications. Paraxylene is used to form dicarboxylic acids and esters, which are key chemical feedstocks in the production of polyesters and aromatics-based plasticizers. Most conventional production routes for these materials utilize fossil fuel-derived feeds. Thus, it would be desirable to find additional synthesis routes for paraxylene and other aromatics that are sustainable, while also providing high-purity end products. Advantageously, the manufacture of such components can be carried out with existing equipment and facilities.

### SUMMARY

In one aspect, the present technology concerns a process for producing a recycled content organic chemical compound (r-organic chemical compound), said process comprising: (a) oxidizing a recycled content paraxylene (r-paraxylene) stream in an oxidation zone of a terephthalic acid (TPA) production facility to provide a recycled content crude terephthalic acid (r-CTA) slurry, wherein the r-paraxylene stream includes recycled content derived from waste plastic; (b) treating at least a portion of the r-CTA slurry to provide a treated r-CTA slurry; and (c) crystallizing at least a portion of the treated r-CTA slurry to provide recycled content purified terephthalic acid (r-PTA).

In one aspect, the present technology concerns a process for producing a recycled content organic chemical compound (r-organic chemical compound), said process comprising: (a) converting waste plastic in at least one conversion facility to form a recycled content aromatics (r-aromatics) stream comprising recycled content paraxylene (r-pX); (b) separating at least a portion of the r-aromatics stream in an aromatics complex to provide a recycled content paraxylene (r-paraxylene) stream; (c) oxidizing at least a portion of the r-paraxylene stream in an oxidation zone of a terephthalic acid production facility to provide a recycled content crude terephthalic acid (r-CTA) slurry; and (d) processing at least a portion of the r-CTA slurry to provide a stream of recycled content purified terephthalic acid (r-PTA).

In one aspect, the present technology concerns a process for producing a recycled content organic chemical compound (r-organic chemical compound), said process comprising: (a) forming recycled content purified terephthalic acid (r-PTA) from a recycled content paraxylene (r-paraxylene) stream in a terephthalic acid (TPA) production facility, wherein the r-paraxylene stream includes recycled content derived from waste plastic; and (b) reacting at least a portion of the r-PTA with ethylene glycol (EG) to form recycled content polyethylene terephthalate (r-PET).

In one aspect, the present technology concerns a process for producing a recycled content organic chemical compound (r-organic chemical compound), said process comprising: reacting re a stream comprising recycled content purified terephthalic acid (r-PTA) with ethylene glycol (EG) to form a recycled content polyethylene terephthalate (r-PET), wherein the r-PTA includes recycled content from mixed waste plastic.

### BRIEF DESCRIPTION OF THE DRAWINGS

FIG. 1a is a block flow diagram illustrating the main steps of a process for making recycled content aromatics (r-aromatics) and recycled content paraxylene (r-paraxylene), and optionally, a recycled content organic chemical compound from the r-paraxylene, wherein the r-aromatics (and r-paraxylene and r-organic chemical compound) have physical content from one or more source materials;

FIG. 1b is a block flow diagram illustrating the main steps of a process for making recycled content aromatics (r-aromatics) and recycled content paraxylene (r-paraxylene), and optionally, a recycled content organic chemical compound from the r-paraxylene, wherein the r-aromatics (and r-paraxylene and r-organic chemical compound) have credit-based recycled content from one or more source materials;

FIG. 2 is a schematic block flow diagram illustrating the main processes/facilities in a system for providing recycled content organic chemical compounds, including r-paraxylene, r-terephthalic acid, and r-polyethylene terephthalate, according to various embodiments of the present invention;

FIG. 3a is a schematic block flow diagram illustrating the main processes/facilities in the system shown in FIG. 2 and particularly illustrating additional processing schemes for producing recycled content products;

FIG. 3b is a schematic block flow diagram of the facility shown in FIG. 2, particularly illustrating additional processing steps for producing recycled content intermediates for use in forming the recycled content products; and

FIG. 4 is a schematic block flow diagram illustrating the main steps/zones of a TPA production facility and a PET production facility suitable for use in the system illustrated in FIG. 2.

### DETAILED DESCRIPTION

We have discovered new methods and systems for producing paraxylene and organic chemical compounds formed by directly processing paraxylene or its derivatives, including, for example, organic chemical compounds such as terephthalic acid and polyethylene terephthalate. More specifically, we have discovered a process and system for producing paraxylene where recycled content from waste materials, such as waste plastic, are applied to paraxylene (or its derivatives) in a manner that promotes the recycling of waste plastic and provides paraxylene (or other organic chemical compounds) with substantial amounts of recycled content.

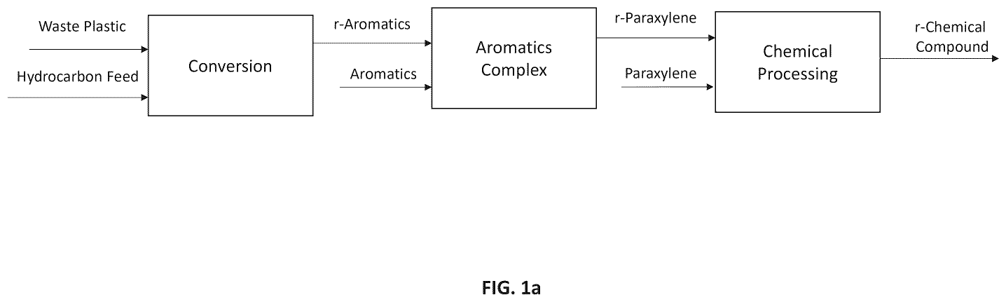

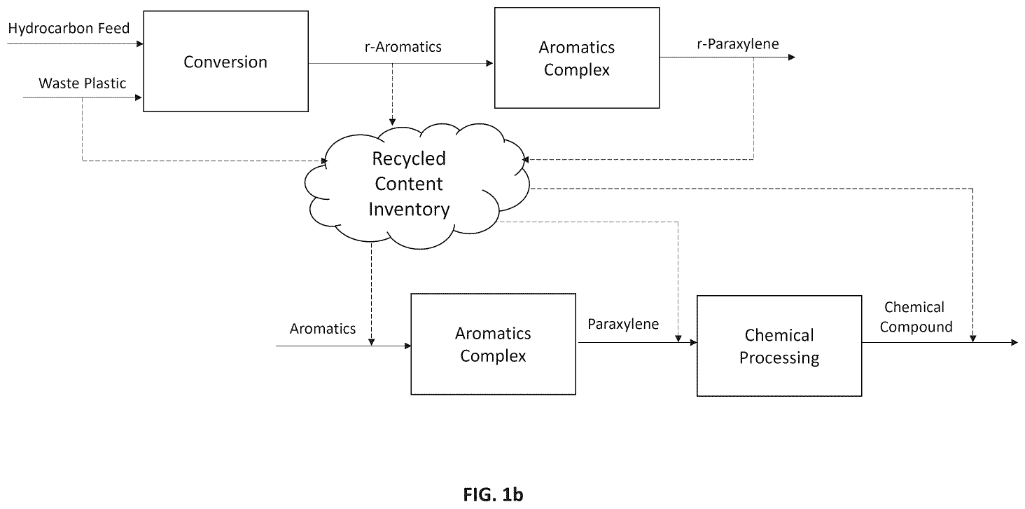

Turning initially to FIGS. 1a and 1b, paraxylene is formed by processing a predominantly aromatics stream in an aromatics complex to provide a stream including at least 85, at least 90, at least 92, at least 95, at least 97, or at least 99 weight percent paraxylene. The paraxylene stream can undergo one or more additional processing steps to provide at least one organic chemical compound derived from paraxylene. Examples of such organic chemical compounds include, but are not limited to, terephthalic acid, polymers such as polyethylene terephthalate, and other related organic chemical compounds.

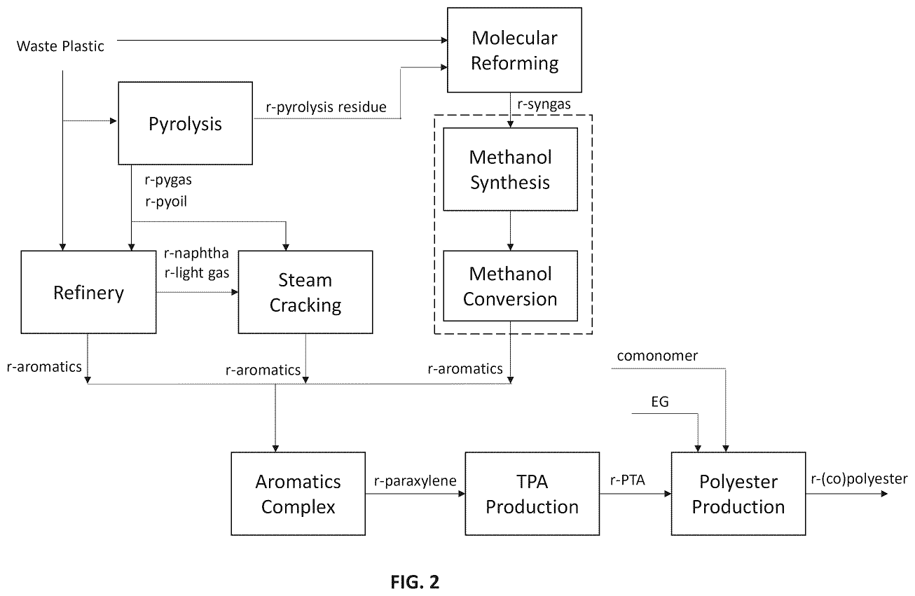

As generally shown in FIGS. 1a and 1b, a stream of waste plastic processed in one or more conversion facilities may provide the aromatics stream, which can be processed to form the paraxylene stream. The recycled content in the paraxylene stream can be physical and may directly originate from waste plastic or an intermediate hydrocarbon stream formed by processing waste plastic (not shown in FIGS. 1 or 2), and/or the recycled content may be credit based and can be applied to a target stream in the aromatics complex and/or chemical processing facilities.

The aromatics (or paraxylene or organic chemical compound) streams can have a total recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. Similarly, the r-TPA and/or r-PET or even the r-aromatics stream can have a recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. The recycled content in one or more of these streams can be physical recycled content, credit-based recycled content, or a combination of physical and credit-based recycled content.

Turning initially to FIG. 1a, in one embodiment or in combination with one or more embodiments mentioned herein, at least a portion of the recycled content in the aromatics and/or paraxylene stream (or in the organic chemical compound product stream) can be physical (direct) recycled content. This recycled content may originate from a waste plastic stream. The waste plastic stream is ultimately converted in one or more conversion facilities (e.g., a pyrolysis facility, a refinery, a steam cracking facility, and/or a molecular reforming facility and methanol-to-aromatics facility), which is processed (alone or with a non-recycled content aromatics stream) as described herein to provide an r-paraxylene stream. The r-paraxylene stream can then be further processed (along or in combination with a non-recycled content paraxylene stream) to provide a recycled content organic chemical compound, including, but not limited to, recycled content terephthalic acid (r-TPA), recycled content polyethylene terephthalate (r-PET), and one or more additional recycled content organic chemical compounds (r-organic chemical compounds).

The amount of physical recycled content in the target product (e.g. composition, r-aromatics or r-paraxylene or r-organic chemical compound) can be determined by tracing the amount of waste plastic material processed along a chain of chemical pathway(s) and ending with the moiety or portion of the target product attributable to the waste plastic chemical pathway. As used herein, a moiety can be a portion the atoms and their structure of a target product and can also include the entire chemical structure of the target product, and does not necessarily require the inclusion of a functional group. For example, a moiety of p-xylene can include the aromatic ring, a portion of the aromatic ring, the methyl groups, or the entire p-xylene molecule. The chemical pathway includes all chemical reactions and other processing steps (e.g., separations) between the starting materials (e.g., waste plastic) and the moiety in the target product attributable to the chemical pathway originating in waste plastics. For example, the chemical pathway for the r-aromatics can include pyrolysis, optionally refining and/or stream cracking, and/or molecular reforming and methanol synthesis and conversion. The chemical pathway for the r-paraxylene can further include processing in the aromatics complex, and the chemical pathway for the r-organic chemical compound may include a variety of additional steps, such as, for example, oxidation, polymerization, etc., depending on the specific r-organic chemical compound. A conversion factor may be associated with each step along the chemical pathway. The conversion factors account for the amount of the recycled content diverted or lost at each step along the chemical pathway. For example, the conversion factors can account for the conversion, yield, and/or selectivity of the chemical reactions along the chemical pathway.

The amount of credit-based recycled content in a target product (e.g. compositions, r-aromatics or r-paraxylene or r-organic chemical compounds) can be determined by calculating the mass weight percent of a target moiety in a target product and attributing a recycle content credit to the target product in any amount up to the mass weight percent of the target moiety in the target product as a maximum. The credit based recycle content that is eligible to be applied to the target product is determined by tracing the waste plastic material along a chain of chemical pathway(s) and ending with the same moiety as target moiety in the target product. Thus, the credit based recycle content can be applied to a variety of different target products having the same moiety even though the products are made by entirely different chemical pathways provided that the credit applied is obtained from waste plastic and the waste plastic ultimately undergoes at least one chemical pathway originating from waste plastic and ending in the target moiety. For example, if a recycle content credit is obtained from waste plastic and booked into a recycle content inventory, and there exists chemical pathways at the facility capable of processing the waste plastic through to a target moiety such as p-xylene (e.g. a pyrolysis reactor effluent to a crude distillation column to a hydrotreater to a reformer to an aromatics complex that isolates p-xylene), the recycle content credit is then a type eligible to apply to any p-xylene molecule made by any chemical pathway, including the one existing at the facility and/or to the p-xylene portion of a pyrolysis gasoline stream composition obtained from a steam cracker and gasoline fractionator. As with physical recycled content, a conversion factor may or may not be associated with each step along the chemical pathway. Additional details on credit-based recycled content are provided below.

The amount of recycled content applied to the r-aromatics (or r-paraxylene or r-organic chemical compound) can be determined using one of variety of methods for quantifying, tracking, and allocating recycled content among various materials in various processes. One suitable method, known as “mass balance,” quantifies, tracks, and allocates recycled content based on the mass of the recycled content in the process. In certain embodiments, the method of quantifying, tracking, and allocating recycled content is overseen by a certification entity that confirms the accuracy of the method and provides certification for the application of recycled content to the r-aromatics (or r-paraxylene or r-organic chemical compound).

Turning now to FIG. 1b, one embodiment where the r-organic chemical compound (or r-paraxylene) includes credit-based recycled content, is provided. Recycled content credits from waste plastic are attributed to one or more streams within the facility. For example, the recycled content credits derived from waste plastics can be attributed to the aromatics stream fed to the aromatics complex, or to any of the products separated and isolated in the aromatics complex, such as to the paraxylene stream. Alternatively, or in addition, recycled content credits obtained from one or more intermediate streams within the conversion facility and/or aromatics complex can also be attributed to one or more products, such as paraxylene, within the facility, depending on the specific configuration of the system. Further, recycled content credits from one or more of these streams may also be attributed to the organic chemical compound stream, as shown in FIG. 1b.

As such, the waste plastic stream, or the r-aromatics stream and r-paraxylene streams (and any recycled content intermediate streams not shown in FIG. 1b) not made at the facility or purchased or acquired, can each act as a “source material” of recycled content credits. The aromatics fed to the aromatics complex, the paraxylene product or any other products separated and/or isolated from the aromatics complex, the paraxylene transferred (including sales) or fed to the chemical processing facility, any intermediate streams not shown, and even the organic chemical compound, can each act as a target product to which the recycled content credits are attributed. In one embodiment or in combination with any embodiment mentioned herein, the source material has physical recycled content and the target product has less than 100 percent physical recycled content. For example, the source material can have at least 10, at least 25, at least 50, at least 75, at least 90, at least 99, or 100 percent physical recycled content and/or the target product can have less than 100, less than 99, less than 90, less than 75, less than 50, less than 25, less than 10, less than 1 percent, or no physical recycled content.

The ability to attribute recycled content credits from a source material to a target product removes the co-location requirement between the facility making the source material (with physical recycled content) and the facility making the aromatics or products receiving recycle content value (e.g. paraxylene or organic chemical compound). This allows a chemical recycling facility/site in one location to process waste material into one or more recycled content source materials and then apply recycled content credits from those source materials to one or more target products being processed in existing commercial facilities located remotely from the chemical recycling facility/site, optionally within the same Family of Entities, or to associate a recycle content value with a product that is transferred to another facility, optionally owned by a different entity that can deposit the recycle content credit into its recycle content inventory one the product is receiving, purchased, or otherwise transferred. Further, the use of recycled content credits allows different entities to produce the source material and the aromatics (or paraxylene or organic chemical compound). This allows efficient use of existing commercial assets to produce the aromatics (or paraxylene or organic chemical compound). In one or more embodiments, the source material is made at a facility/site that is at least 0.1, at least 0.5, at least 1, at least 5, at least 10, at least 50, at least 100, at least 500, or at least 1000 miles from the facility/site where the target product is used to make the aromatics (or paraxylene or organic chemical compound).

The attributing of recycled content credits from the source material (e.g., the r-aromatics from the conversion facility) to the target product (e.g., an aromatics stream fed to an aromatics complex) can be accomplished by transferring recycled content credits directly from the source material to the target product. Alternatively, as shown in FIG. 1b, recycled content credits can be applied from any of the waste plastic, r-aromatics, and r-paraxylene (when present) to the aromatics, paraxylene, or organic chemical compound via a recycled content inventory.

When a recycled content inventory is used, recycled content credits from the source material having physical recycled content (e.g., the waste plastic, the r-aromatics, and optionally the r-paraxylene shown in FIG. 1b) are booked into the recycled content inventory. The recycled content inventory can also contain recycled content credits from other sources and from other time periods. In one embodiment, recycled content credits in the recycled content inventory correspond to a moiety, and the recycle content credit is applied or assigned to the same a target products containing a target moiety, and the target moiety is either (i) not chemically traceable through chemical pathways used to for generating the recycle content credit or (ii) is chemically traceable through chemical pathways used for generating the recycle content credit. Chemical traceability is achieved when atoms from a source material such as waste plastic can be theoretically traced to one or more atoms in the target moiety of a target product through each chemical pathway to obtain that atom(s) in the target moiety.

In some embodiments, there may be a periodic (e.g., annual or semi-annual) reconciliation between waste plastic credits deposited in the recycled content inventory and the mass of waste plastic processed. Such reconciliations may be performed by an appropriate entity at an interval consistent with rules of the certification system in which the producer is participating.

In one embodiment, once recycled content credits have been attributed to the target product (e.g., the aromatics stream, the paraxylene stream, or any intermediate stream not shown), the amount of the credit-based recycled content allocated to the organic chemical compound (e.g., TPA, PET, or other organic chemical compound) is calculated by the mass proportion of atoms in the target product that are chemically traceable to the source material. In another embodiment, a conversion factor can be associated with each step along the chemical pathway of the credit-based recycled content. The conversion factors account for the amount of the recycled content diverted or lost at each step along the chemical pathway. For example, the conversion factors can account for the conversion, yield, and/or selectivity of the chemical reactions along the chemical pathway. However, if desired, the amount of recycle content applied to a target product can be more than the mass proportion of the target moiety chemically traceable to the waste plastic source material. The target product can receive up to 100% recycle content even though the mass proportion of atoms in the target moiety that is chemically traceable to a recycle source material, such as mixed plastic waste stream, is less than 100%. For example, if the target moiety in a product represents only 30 wt. % of all atoms in a target product that are chemically traceable to a mixed plastic waste stream, the target product can nevertheless receive more than 30% recycle content value, up to 100% if desired. While such application would violate chemical traceability for the full value of the amount of recycle content in a target product back to a waste plastic source, the particular amount of recycle content value applied to a target product will depend on the rules of a certification system that the producer participates in.

As with the physical recycled content, the amount of credit-based recycled content applied to the r-aromatics (or r-paraxylene or r-organic chemical compound) can be determined using one of variety of methods, such as mass balance, for quantifying, tracing, and allocating recycled content among various products in various processes. In certain embodiments the method of quantifying, tracing, and allocating recycled content is overseen by a certification entity that confirms the accuracy of the method and provides certification for the application of recycled content to the r-aromatics (or r-paraxylene or r-organic chemical compound).

The r-aromatics (or r-paraxylene or r-organic chemical compound) can have 25 to 90, 40 to 80, or 55 to 65 percent credit-based recycled content and less than 50, less than 25, less than 10, less than 5, or less than 1 percent physical recycled content. In certain embodiments, the r-aromatics (or r-paraxylene or r-organic chemical compound) can have at least 10, at least 25, at least 50, or at least 65 percent and/or not more than 90, not more than 80, or not more than 75 percent credit-based recycled content from one or more of the r-aromatics and/or r-paraxylene, individually.

In one or more embodiments, the recycled content of the r-aromatics (or r-paraxylene or r-organic chemical compound) can include both physical recycled content and credit-based recycled content. For example, the r-aromatics (or r-paraxylene or r-organic chemical compound) can have at least 10, at least 20, at least 30, at least 40, or at least 50 percent physical recycled content and at least 10, at least 20, at least 30, at least 40, or at least 50 percent credit-based recycled content. As used herein, the term “total recycled content” refers to the cumulative amount of physical recycled content and credit-based recycled content from all sources.

Turning now to FIG. 2, a process and facility for use in forming a recycled content organic chemical compound (r-organic chemical compound) is provided. As used herein, the term “organic chemical compound,” refers to a chemical compound that includes carbon and hydrogen atoms, but also includes oxygen and/or nitrogen atoms. An organic chemical compound can include at least 75, at least 80, at least 85, at least 90, at least 95, or at least 99 atom percent of carbon and hydrogen atoms combined, with the balance being nitrogen and oxygen.

Specifically, the system shown in FIG. 2 illustrates several types of waste plastic conversion facilities (e.g., a pyrolysis facility, a refinery, a steam cracking facility, a molecular reforming facility and related methanol-to-aromatics conversion facilities, and an optional PET cracking facility) for processing a stream of waste plastic (and/or one or more streams derived from waste plastic) to provide streams of recycled content aromatics (r-aromatics). Additionally, although not shown in FIG. 2, each of these conversion facilities may also process a conventional stream of hydrocarbon-containing materials along with the waste plastic and/or stream derived from waste plastic. For example, the refinery may also process crude oil, the steam cracking facility may also process a hydrocarbon stream (e.g., light gas and/or naphtha), and the molecular reforming facility may also process at least one hydrocarbon-containing stream (e.g., coal, petroleum, etc.) Further, the aromatics complex may also receive and process another aromatics-containing stream not from one or more of the conversion facilities. These additional feed streams may or may not include recycled content.

As shown in FIG. 2, the r-aromatics stream or stream from one or more of the conversion facilities may then be further processed in an aromatics complex to provide recycled content paraxylene (r-paraxylene), which can then be oxidized to form recycled content terephthalic acid (r-TPA) in a TPA production facility. Optionally, at least a portion of the r-PTA can be further reacted to form recycled content polyethylene terephthalate (r-PET) in PET production facility. One or more of the recycled content streams from these facilities can be used in other applications not specifically shown in or discussed with respect to FIG. 2.

The system shown in FIG. 2 can include or be a chemical recycling facility. Chemical recycling facilities are not the same as mechanical recycling facilities. As used herein, the terms “mechanical recycling” and “physical recycling” refer to a recycling process that includes a step of melting waste plastic and forming the molten plastic into a new intermediate product (e.g., pellets or sheets) and/or a new end product (e.g., bottles). Generally, mechanical recycling does not substantially change the chemical structure of the plastic being recycled. The chemical recycling facilities described herein may be configured to receive and process waste streams from and/or that are not typically processable by a mechanical recycling facility.

In one embodiment or in combination with any embodiments mentioned herein, at least two, at least three, at least four, at least five, at least six, at least seven, or all of the pyrolysis facility, the refinery, the steam cracking facility, the molecular reforming facility, the methanol-to-aromatics conversion facility, the aromatics complex, and the TPA production facility and the PET production facility may be co-located. As used herein, the term “co-located” refers to the characteristic of at least two objects being situated on a common physical site, and/or within 5, within 3, within 1, within 0.75, within 0.5, or within 0.25 miles of each other, measured as a straight-line distance between two designated points. When two or more facilities are co-located, the facilities may be integrated in one or more ways. Examples of integration include, but are not limited to, heat integration, utility integration, waste-water integration, mass flow integration via conduits, office space, cafeterias, integration of plant management, IT department, maintenance department, and sharing of common equipment and parts, such as seals, gaskets, and the like.

Additionally, one or more, two or more, three or more, four or more, five or more six or more, seven, or all, of the pyrolysis facility, the refinery, the steam cracking facility, the molecular reforming facility, the methanol-to-aromatics conversion facility, the aromatics complex, the TPA production facility, and the PET production facility may be commercial-scale facilities. For example, in one embodiment or in combination with any embodiments mentioned herein, one or more of these facilities/steps can accept one or more feed streams at a combined average annual feed rate of at least 500, at least 1000, at least 1500, at least 2000, at least 5000, at least 10,000, at least 50,000, or at least 100,000 pounds per hour, averaged over one year. Further, one or more of the facilities can produce at least one recycled content product streams at an average annual rate of at least 500, or at least 1000, at least 1500, at least 2000, at least 2500, at least 5000, at least 10,000, at least 50,000, or at least 75,000 pounds per hour, averaged over one year. When more than one r-product stream is produced, these rates can apply to the combined rate of all r-products.

One or more, two or more, three or more, four or more, five or more, six or more, seven, or all, of the pyrolysis facility, the refinery, the steam cracking facility, the molecular reforming facility, the methanol-to-aromatics conversion facility, the aromatics complex, the TPA production facility, and the PET production facility can be operated in a continuous manner. For example, each of the steps or processes within each of the facilities and/or the process amongst the facilities may be operated continuously and may not include batch or semi-batch operation. In one embodiment or in combination with any embodiments mentioned herein, at least a portion of one or more of the facilities may be operated in a batch or semi-batch manner, but the operation amongst the facilities may be continuous overall.

As shown in FIG. 2, waste plastic may be introduced into one or more conversion facilities for processing the waste plastic (or hydrocarbon streams derived from waste plastic) to form recycled content products. Examples of conversion facilities shown in FIG. 2 include a pyrolysis facility, a refinery, a steam cracking facility, a molecular reforming facility (with methanol-to-aromatics conversion facility), and a PET cracking facility. A single chemical recycling complex may include one or more of these facilities or several conversion facilities may be in separate locations (i.e., not co-located). As shown in FIG. 2, these facilities may work independently or in combination to provide a stream of recycled content aromatics (r-aromatics), which can then be processed to form recycled content terephthalic acid (r-PTA) and, in some cases, recycled content polyethylene terephthalate (r-PET). The basic operation of these facilities will now be discussed in further detail below.

In one embodiment or in combination with any embodiment mentioned here, a stream of mixed waste plastic may be passed through a plastics processing facility (not shown) and the processed waste plastic may be introduced into one or more of the conversion units. The plastics processing facility, when present, can separate mixed plastic into PET-enriched and a polyolefin (PO)-enriched streams and these separated streams can be introduced into separate conversion facilities. Additionally, or in the alternative, the plastics processing facility may also reduce the size of the incoming plastic via a crushing, flaking, pelletizing, grinding, granulating, and/or pulverizing step and/or the waste plastic may be melted or combined with a liquid to form liquified plastic or a slurry. One or more cleaning or separation steps may also be present to remove dirt, food, sand, glass, aluminum, lignocellulosic materials such as paper and cardboard, from the incoming waste stream.

Turning initially to the pyrolysis facility, waste plastic (and, in some cases, predominantly PO-containing waste plastic) may be introduced into the pyrolysis facility, wherein it can be pyrolyzed to form at least one recycled content pyrolysis effluent (r-pyrolysis effluent) stream. Any suitable pyrolysis facility/step can be used and it may include, for example, at least one pyrolysis reactor for chemically and/or thermally decomposing the waste plastic. Although pyrolysis is generally carried out in a reaction environment that is substantially free of molecular oxygen, pyrolysis processes may be further defined by other parameters such as the pyrolysis reaction temperature within the reactor, the residence time in the pyrolysis reactor, the reactor type, the pressure within the pyrolysis reactor, and the presence or absence of pyrolysis catalysts.

The feed to the pyrolysis reactor can comprise, consists essentially of, or consists of waste plastic, and the feed stream can have a number average molecular weight (Mn) of at least 3000, at least 4000, at least 5000, or at least 6000 g/mole. If the feed to the pyrolysis reactor contains a mixture of components, the Mn of the pyrolysis feed is the average Mn of all feed components, based on the weight of the individual feed components. The waste plastic in the feed to the pyrolysis reactor can include post-consumer waste plastic, post-industrial waste plastic, or combinations thereof. In certain embodiments, the feed to the pyrolysis reactor comprises less than 5, less than 2, less than 1, less than 0.5, or about 0.0 weight percent coal and/or biomass (e.g., lignocellulosic waste, switchgrass, fats and oils derived from animals, fats and oils derived from plants, etc.). The feed to the pyrolysis reaction can also comprise less than 5, less than 2, less than 1, or less than 0.5, or about 0.0 weight percent of a co-feed stream, including steam and/or sulfur-containing co-feed streams. In other cases, steam fed to the pyrolysis reactor can be present in amounts of up to 50 weight percent.

The pyrolysis reaction can involve heating and converting the waste plastic feedstock in an atmosphere that is substantially free of molecular oxygen or in an atmosphere that contains less molecular oxygen relative to ambient air. For example, the atmosphere within the pyrolysis reactor may comprise not more than 5, not more than 4, not more than 3, not more than 2, not more than 1, or not more than 0.5 weight percent of molecular oxygen. The pyrolysis reaction in the reactor can be thermal pyrolysis, which is carried out in the absence of a catalyst, or catalytic pyrolysis, which is carried out in the presence of a catalyst. When a catalyst is used, the catalyst can be homogenous or heterogeneous and may include, for example, oxides, certain types of zeolites and other mesostructured catalysts.

The pyrolysis reactor may have any suitable design and can comprise a film reactor, a screw extruder, a tubular reactor, a stirred tank reactor, a riser reactor, a fixed bed reactor, a fluidized bed reactor, a rotary kiln, a vacuum reactor, a microwave reactor, or an autoclave. The reactor may also utilize a feed gas and/or lift gas for facilitating the introduction of the feed into the pyrolysis reactor. The feed gas and/or lift gas can comprise nitrogen and can comprise less than 5, less than 2, less than 1, or less than 0.5, or about 0.0 weight percent of steam and/or sulfur-containing compounds. The feed and/or lift can also include light hydrocarbons, such a methane, or hydrogen, and these gases may be used alone or in combination with steam.

After exiting the reactor, a stream of recycled content pyrolysis effluent (r-pyrolysis effluent) can be separated to form recycled content pyrolysis streams, including recycled content pyrolysis residue (r-pyrolysis residue) and recycled content pyrolysis vapor (r-pyrolysis vapor), or the r-pyrolysis vapor may be further separated to provide streams of recycled content pyrolysis gas (r-pygas) and recycled content pyrolysis oil (r-pyoil). In some cases, the second separation step may be omitted so that the stream of r-pyrolysis vapor is removed from the facility and introduced into a downstream processing facility.

When withdrawn as a separate product stream, the r-pyoil can include predominantly C5 to C22 hydrocarbon components, or it can include at least 55, at least 60, at least 65, at least 70, at least 75, or at least 80 weight percent of C5 to C22 hydrocarbon components, while the r-pygas can include predominantly C2 to C4 hydrocarbon components, or at least 30, at least 40, at least 45, at least 55, at least 60, at least 65, at least 70, at least 75, or at least 80 weight percent C2 to C4 hydrocarbon components. In some cases, the C2 to C4 components in the r-pygas can include at least 50, at least 55, at least 60, at least 65, at least 70, or at least 75 weight percent of alkanes and/or at least 40, at least 45, at least 50, at least 55, at least 60, at least 65, at least 70, or at least 75 weight percent of olefins, based on the amount of C2 to C4 hydrocarbon components in the stream. The r-pyrolysis residue stream may include at least 55, at least 65, at least 75, at least 85, or at least 90 weight precent of C20 and heavier hydrocarbons (e.g., pyrolysis waxes), as well as carbon-containing components that are solid at 200° C. and 1 atmosphere, absolute (e.g., pyrolysis char).

The r-pyoil may also comprise one or more of the following (i) through (v): (i) less than 500 ppm, less than 450 ppm, less than 350 ppm, less than 250 ppm, less than 100 ppm, less than 75 ppm, less than 50 ppm, less than 25 ppm, or less than 10 ppm sulfur; (ii) less than 300 ppm, less than 150 ppm, less than 100 ppm, less than 50 ppm, less than 25 ppm, less than 10 ppm, or less than 5 ppm chlorine; (iii) less than 500 ppm, less than 250 ppm, less than 100 ppm, less than 75 ppm, less than 50 ppm, less than 30 ppm, or less than 20 ppm water; (iv) less than 500 ppb, less than 250 ppb, less than 100 ppb, less than 50 ppb, less than 25 ppb, less than 10 ppb, less than 5 ppb, or less than 2 ppb arsenic; and/or (v) less than 1500 ppm, less than 1000 ppm, less than 500ppm, less than 250 ppm, less than 100 ppm, less than 75 ppm, less than 50 ppm, less than 30 ppm, or less than 20 ppm nitrogen.

As shown in FIG. 2, at least a portion of the r-pyrolysis residue may be introduced into the molecular reforming facility alone or in combination with a stream of waste plastic and/or other feed stream (not shown), which may or may not include recycled content. Examples of the other feed stream introduced into the molecular reforming facility can include, but are not limited to, coal, petroleum coke, lignocellulosic materials, liquid hydrocarbons, natural gas, organic hydrocarbons, and mixtures thereof. When introduced into the molecular reforming facility, the waste plastic can be in the form of a solid powder and/or in the form of a slurry with water or other liquid.

As used herein, the term “molecular reforming” refers to conversion of a carbon-containing feed into syngas (CO, CO2, and H2). Molecular reforming encompasses both steam reforming and partial oxidation (POX) gasification. As used herein, the term “steam reforming” refers to the conversion of a carbon-containing feed into syngas (i.e., a gas stream comprising at least 90, at least 95, at least 97, or at least 99 weight percent carbon monoxide, hydrogen, and carbon dioxide) via reaction with water. The steam reforming can include, for example, steam-methane reforming in which the carbon-containing feed includes a methane-containing stream, such as natural gas. As used herein, the term “partial oxidation (POX) gasification” or “POX gasification” refers to high temperature conversion of a carbon-containing feed into syngas, where the conversion is carried out in the presence of a less-than-stoichiometric amount of oxygen. The carbon-containing feed to POX gasification can include solids, liquids, and/or gases and may, in some cases, include waste plastic. When one or more feed streams to the molecular reforming facility include waste plastic or recycled content derived from waste plastic (or another source), the syngas produced is recycled content syngas (r-syngas). The r-syngas may further include non-recycled content when a portion of the feed does not include or is not derived from waste plastic.

As shown in FIG. 2, at least a portion of the r-syngas formed in the molecular reforming facility may be introduced into a methanol-to-aromatics conversion facility. The methanol-to-aromatics (MTA) conversion facility includes a methanol synthesis step for synthesizing methanol from syngas (or synthesizing recycled content methanol, r-methanol, from r-syngas) and a methanol conversion step for converting r-methanol to a stream of recycled content aromatics (r-aromatics). In some cases, the MTA conversion facility may first react a stream of methanol (or r-methanol) at temperatures of around 400 to 600° C., or 450 to 500° C., over a selective catalyst (e.g.,

ZSM) to form a mixture of aromatics, olefins, and alkanes. Some of the heavier alkanes and/or olefins can be recycled, along with at least a portion of the benzene and/or toluene, to increase conversion, while the lighter alkanes may be further reacted at a higher temperature of 500 to 600° C. to form additional aromatics (r-aromatics), which can be further processed (e.g., separated) to provide a recycled content aromatics (r-aromatics) stream as shown in FIG. 2. The resulting r-aromatics stream exiting the methanol-to-aromatics conversion facility can include recycled content benzene, toluene, and xylenes (r-BTX), and may include, for example, at least 35, at least 40, at least 45, or at least 50 weight percent and/or not more than 95, not more than 85, not more than 75, not more than 70, not more than 65, or not more than 60 weight percent of these components.

Turning back to FIG. 2, when the chemical recycling facility includes a refinery, at least a portion of the r-pyoil and/or r-pygas (or r-pyrolysis vapor if not separated in the pyrolysis facility) may be introduced into one or more locations of the refinery to undergo at least one processing step to provide one or more recycled content hydrocarbon products. Examples of recycled content hydrocarbon products produced by the refinery can include, but are not limited to, recycled content light gas (r-light gas), recycled content naphtha (r-naphtha), and recycled content aromatics (r-aromatics). Additionally, a stream of waste plastic, typically liquified waste plastic, can also be processed in at least one unit within the refinery to provide these recycled content streams.

The processing steps utilized in the refinery can include separation or distillation, cracking, and reforming, along with other processing steps for removing sulfur, nitrogen, and other impurities. In some cases, the r-pyoil and/or r-pyrolysis vapor can be introduced into an atmospheric distillation column (ADU) and can be separated along with a crude oil feedstock to form several recycled content hydrocarbon fractions. Lighter fractions, such as r-light gas, can be further separated to remove impurities, while heavier fractions, such as r-gas oils, can be introduced into gas oil crackers and thermally and/or catalytically cracked to provide recycled content cracked light gas (r-cracked light gas) and recycled content cracked naphtha (r-cracked naphtha). At least a portion of the r-cracked naphtha, along with the r-naphtha removed from the ADU, can be introduced into a reformer unit, wherein it can be converted to a recycled content reformate (r-reformate) stream. The r-reformate stream may comprise predominantly C6 to C10 aromatics and at least a portion of this stream can be withdrawn from the refinery as the r-aromatics stream shown in FIG. 2.

When the chemical recycling complex includes a steam cracking facility, at least a portion of the r-light gas and/or r-naphtha from the refinery and/or the r-pygas and/or r-pyoil from the pyrolysis facility can be introduced into the steam cracking facility. In some cases, gas-phase streams (e.g., r-pygas and/or r-light gas, optionally with another predominantly C2 to C4 gas stream with or without recycled content) can be introduced into the inlet of a steam cracker furnace in the steam cracking facility, while, in other cases, these streams may be introduced into one or more locations downstream of the furnace. When one or more liquid-phase streams (e.g., r-pyoil and/or r-naphtha, optionally with another predominantly C5 to C22 liquid stream with or without recycled content) are introduced into the steam cracking facility, these streams can be fed to the inlet of the steam cracking furnace.

In the steam cracking furnace, the hydrocarbon feed stream, which can include one or more of r-pygas, r-pyoil, r-light gas, and r-naphtha, can be thermally cracked in the presence of steam to form a predominantly recycled content olefin-containing (r-olefin-containing) stream and a stream of recycled content pyrolysis gasoline (r-pyrolysis gasoline). The r-olefin-containing stream may be compressed and further processed in a separation zone of the steam cracking facility to provide one or more recycled content olefin (r-olefin) products (e.g., r-ethylene and/or r-propylene), while a recycled content pyrolysis gasoline (r-pyrolysis gasoline), which comprises predominantly C6 to C10 aromatics, can be withdrawn from the steam cracking facility as the r-aromatics stream shown in FIG. 2.

The r-aromatics stream or streams withdrawn from each of the refinery, the steam cracking facility, the MTA conversion facility (or two or more, or all of these facilities combined) can have one or more of the following properties (i) through (viii): (i) the stream(s) can comprise predominantly C6 to C10 (or C6 to C9) aromatics, or it can include at least 25, at least 35, at least 45, at least 55, at least 60, at least 65, at least 70, at least 75, at least 80, at least 85, or at least 90 weight percent of C6 to C10 (or C6 to C9) aromatic components; (ii), the stream(s) can comprise less than 75, less than 65, less than 55, less than 45, less than 35, less than 25, less than 15, or less than 10 weight percent of non-aromatic components; (iii) the streams can comprise at least 1, at least 2, at least 3, at least 5, or at least 10 and/or not more than 30, not more than 25, not more than 20, not more than 15, not more than 10, or not more than 7weight percent of benzene, which may include recycled content benzene (r-benzene) and/or non-recycled content benzene; (iv) the stream(s) can comprise at least 5, at least 10, at least 15, or at least 20 and/or not more than 40, not more than 35, not more than 30, not more than 25, or not more than 20 weight percent of toluene, which can include both recycled content toluene (r-toluene) and/or non-recycled content toluene; (v) the stream(s) can comprise at least 2, at least 5, at least 10, at least 15, at least 20, or at least 25 weight percent and/or not more than 75, not more than 70, not more than 65, not more than 60, not more than 55, not more than 50, not more than 45, not more than 40, not more than 35, not more than 30, or not more than 25 weight percent of one or more of C8 aromatics (or recycled content C8 aromatics, r-C8 aromatics), C9 aromatics (or recycled content C9 aromatics, r-C9 aromatics), and C10 aromatics (or recycled content C10 aromatics, r-C10 aromatics, individually or in combination; (vi) the stream(s) can comprise at least 5, at least 10, or at least 15 and/or not more than 50, not more than 45, or not more than 40 weight percent of mixed xylenes, including recycled and non-recycled content xylenes; (vii) the stream(s) may comprise not more than 15, not more than 10, not more than 5, not more than 2, or not more than 1 weight percent of C5 and lighter components and/or C11 and heavier components; and (viii) the stream(s) can comprise a total amount of C6 to C10 (or C9 to C10) hydrocarbon components of at least 55, at least 60, at least 65, at least 70, at least 75, at least 80, at least 85, or at least 90 weight percent, based on the total weight of the stream

Examples of C8 aromatics include, but are not limited to, mixed xylenes such as ortho-xylene, paraxylene, and meta-xylene, as well as ethylbenzene, and styrene, while C9 aromatics can include, for example, isopropyl benzene, propyl benzene, isomers of methyl ethyl benzene, isomers of methyl styrenes, and isomers of trimethyl benzene. Examples of C10 aromatics can include, but are not limited to, isomers of butyl benzene, isomers of diethyl benzene, and isomers of dimethyl ethyl benzene. One or more of these components, when present in the aromatics stream, may include recycled content and/or may include non-recycled content.

In one embodiment or in combination with any embodiment mentioned herein, the r-aromatics stream may comprise 20 to 80, or 25 to 75, or 30 to 60 weight percent benzene and/or 0.5 to 40, or 1 to 35, or 2 to 30 weight percent toluene, and/or 0.05 to 30, or 0.10 to 25, or 0.20 to 20 weight percent of C8 aromatics, based on the total weight of aromatics in the r-aromatics stream.

As shown in FIG. 2, at least a portion of one or more or all of the r-aromatics streams from the conversion facilities (e.g., refinery, steam cracking facility, and/or MTA conversion facility) can be introduced into an aromatics complex, wherein it can be processed to form at least one recycled content aromatics product stream. The r-aromatics stream introduced into the aromatics complex can undergo several processing steps, including, but not limited to, separation (e.g., distillation, extraction, crystallization, adsorption, and combinations thereof), isomerization, alkylation, and transalkylation/disproportionation. The resulting recycled content aromatic products withdrawn from the aromatics complex can include, for example, recycled content paraxylene (r-paraxylene), recycled content metaxylene (r-metaxylene), and recycled content orthoxylene (r-orthoxylene), as well as streams comprising predominantly recycled content benzene (r-benzene), recycled content toluene (r-toluene), and even recycled content C9 and heavier aromatics (r-C9+). In some cases, each of these streams can include at least 55, at least 60, at least 65, at least 70, at least 75, at least 80, at least 85, at least 90, at least 92, or at least 95 weight percent of the named component, based on the total weight of the stream.

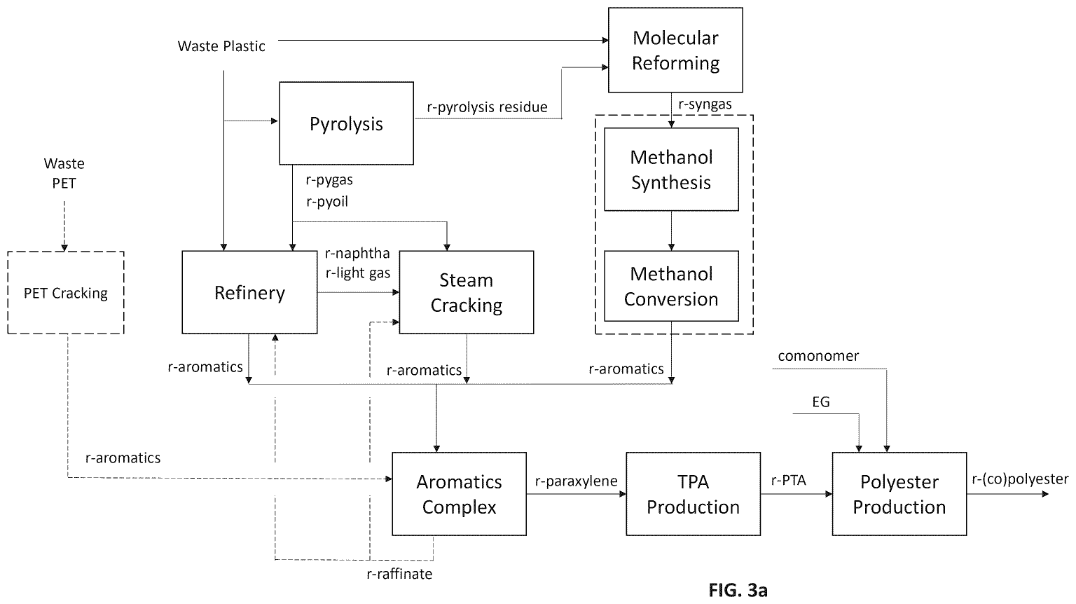

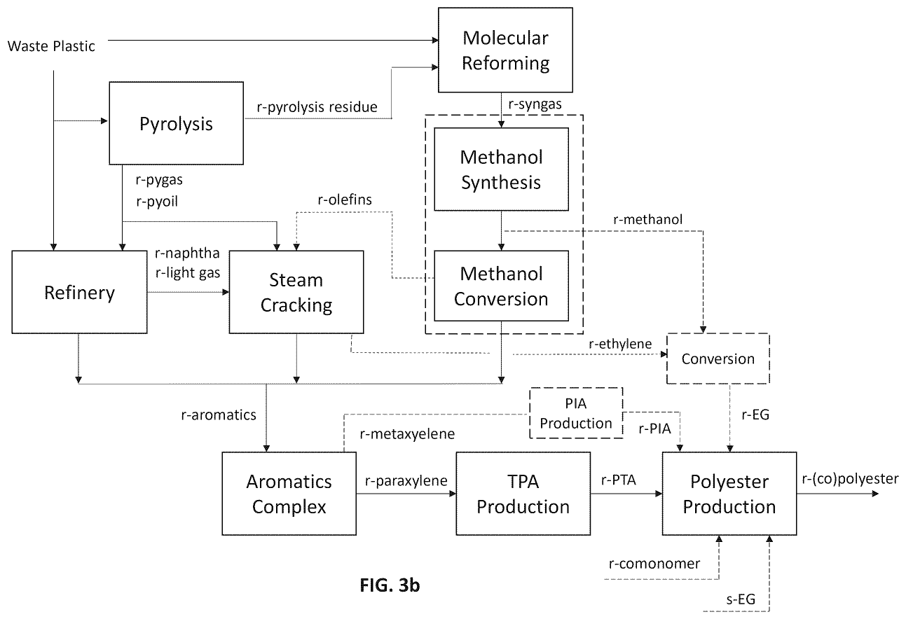

In one embodiment or in combination with any embodiment mentioned herein, a stream of recycled content raffinate (r-raffinate) can be withdrawn from the aromatics complex, as shown by the dashed lines in FIG. 3a. The r-raffinate can comprise predominantly C5 to C8 hydrocarbon components but may include less than 20, less than 15, less than 10, or less than 5 weight percent of aromatics. This stream may be returned to the reformer unit in the refinery and/or into the steam cracker furnace of the steam cracking facility to be further processed to form additional streams including recycled content aromatics (r-aromatics).

In one embodiment or in combination with any embodiment mentioned herein and as generally illustrated in FIG. 3a, the chemical recycling facility may include a PET cracking facility for thermally cracking waste plastic comprising predominantly PET. The feed to the PET cracking facility may include at least 55, at least 60, at least 65, at least 70, at least 75, at least 80, at least 85, at least 90, or at least 95 weight percent of waste PET. In the PET cracking facility, at least a portion of the waste PET can be thermally and/or catalytically cracked and the cracked stream may then be separated to form a stream of recycled content aromatics (r-aromatics). The r-aromatics stream from the PET cracking facility can include at least 50, at least 55, at least 60, at least 65, or at least 75 weight percent of paraxylene, at least a portion of which is r-paraxylene. The r-aromatics stream from the PET cracking facility may also comprise less than 40, less than 35, less than 30, less than 25 weight percent of toluene and xylene in combination. In some cases, the r-aromatics stream from the PET cracking facility can be introduced into and processed in the aromatics complex to form a stream of recycled content r-paraxylene, as shown by the dashed lines in FIG. 2.

As shown in FIG. 2, at least a portion of the r-paraxylene stream withdrawn from the aromatics complex can be introduced into a terephthalic acid (TPA) production facility, wherein the paraxylene can be oxidized and further processed to form recycled content purified terephthalic acid (r-PTA). The r-paraxylene stream introduced into the TPA facility can comprise at least 85, at least 90, at least 95, at least 97, or at least 99 weight percent paraxylene and may or may not include non-recycled content.

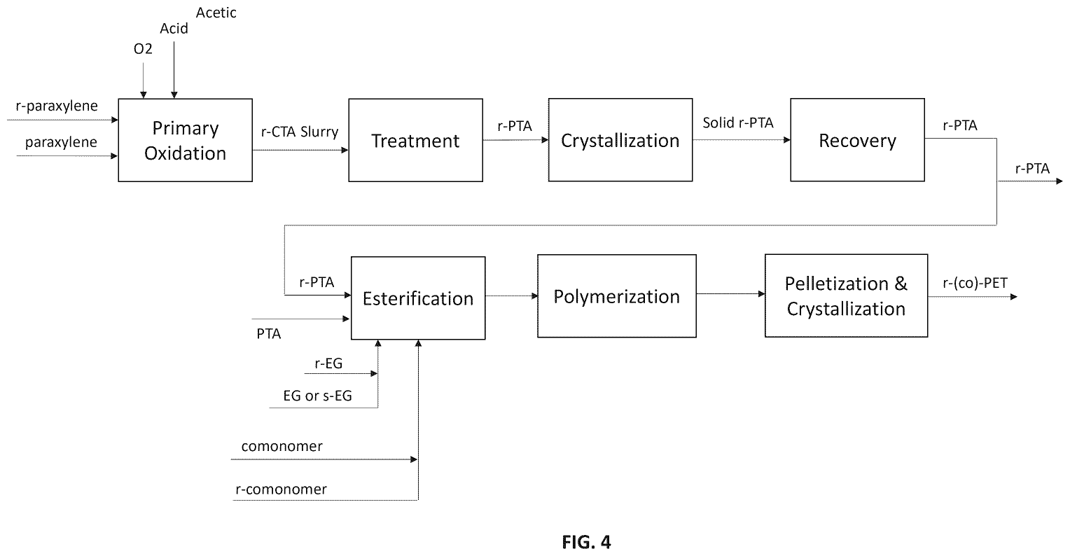

Referring now to FIG. 4, a schematic diagram of the main steps of a TPA production facility for producing r-TPA from r-paraxylene and a PET production facility for producing r-PET from r-TPA are provided. Although shown in the same diagram, the TPA production facility and the PET production facility may or may not be co- located and may or may not be operated by the same commercial entity. Additionally, or in the alternative, the aromatics complex can be integrated with the TPA production facility so that it is located within less than 10, less than 5, less than 2, or less than 1 of the TPA production facility and/or so that it is in fluid flow communication with the TPA production facility.

As shown in FIG. 4, a stream of r-paraxylene (and optionally non-recycled content paraxylene) can be introduced into a primary oxidation step/zone of the TPA production facility, wherein it can be oxidized with molecular oxygen in the presence of a catalyst and a solvent. The catalyst may include several components such as, for example, cobalt, manganese, bromine, and combinations thereof and the solvent can include or be acetic acid. The primary oxidation zone can include at least one reactor that can be operated at a temperature between 120 and 200° C., 140 to about 180° C., or 150 to 170° C. The liquid-phase reaction can be performed in any suitable type of reaction vessel including, but not limited to, CSTRs and bubble columns. The stream withdrawn from the primary oxidation zone comprises a crude terephthalic acid (CTA) slurry and can include a recycled content CTA (r-CTA) slurry when the feed to the primary oxidation step/zone includes r-paraxylene.

The r-CTA slurry includes relatively high levels of impurities, such as 4-carboxybenzaldehyde, para-toluic acid, fluorenones, and other color bodies, that make it unsuitable as a feedstock for the production of PET. Therefore, the r-CTA produced in the primary oxidation step/zone can be subjected to additional treatment that converts the r-CTA into recycled content purified terephthalic acid r-(PTA) suitable for making PET.

As shown in FIG. 4, r-CTA from the primary oxidation step/zone can be further processed to r-PTA via passage through a treatment step/zone, a crystallization step/zone, and a recovery step/zone. In the treatment step/zone, the r-CTA slurry is subjected to additional reactions to remove undesirable impurities and/or color bodies and provide a liquid stream comprising r-PTA.

Examples of suitable methods of removing impurities from the r-CTA slurry include hydrogenation and secondary oxidation. When hydrogenation is used in the treatment step/zone, at least a portion of the initial oxidation solvent (e.g., acetic acid) may be removed from the r-CTA slurry and the r-CTA may be dissolved in water. The resulting aqueous r-CTA stream can then be catalytically hydrogenated to convert the impurities in the r-CTA to more desirable and/or more easily separable compounds. The resulting stream of dissolved r-PTA may be introduced into the crystallization zone, as shown in FIG. 4.

When secondary oxidation is used in the treatment step/zone, at least 55, at least 60, at least 65, at least 70, at least 75, at least 80, at least 85, at least 90, or at least 95 weight percent of the initial acetic acid solvent may be removed from the r-CTA via filtration or other suitable method to provide filtered r-CTA, which can be combined with fresh acetic acid to form a second r-CTA slurry. The second r-CTA slurry may then be further oxidized in a secondary oxidation step/zone to remove/react at least a portion of the impurities and provide an r-PTA slurry. When performed, the secondary oxidation step may be carried out at a higher temperature than the primary oxidation step and may have an average temperature in the range of, for example, about 190° C. to about 280° C., about 200° C. to about 250° C., or about 205° C. to 225° C. The resulting slurry of r-PTA and acetic acid may be introduced into the crystallization zone, as shown in FIG. 4.

The r-PTA stream from the treatment step/zone, whether dissolved in water or formed into an acetic acid slurry, can then be introduced into a crystallization zone, wherein the r-PTA is separated from the liquid to form crystalline r-PTA. When the r-PTA stream is a dissolved r-PTA stream from hydrogenation, the r-PTA can be precipitated from solution, which leaves the water-soluble impurities and other reaction products in solution. Maintaining the solution at a temperature of at least 150° C., or at least 160° C. facilitates easier separation. The r-PTA can then be separated from the liquid via centrifuging and/or filtration to provide solid r-PTA. When the r-PTA stream introduced into the crystallization step is an r-PTA slurry formed by secondary oxidation in acetic acid, the solvent may be separated from the suspended r-PTA particles via centrifuging and/or filtration and the resulting solid r-PTA can be sent to a recovery step/zone as shown in FIG. 4.

In the recovery step/zone, the solid r-PTA can be further processed by cooling, washing and drying, and/or packaging to provide a final r-PTA product. The r-PTA product withdrawn from the recovery step can comprise at least 95, at least 97, at least 99, at least 99.5, at least 99.9, or at least 99.99 weight percent of terephthalic acid (or r-terephthalic acid). The r-PTA product stream may or may not also include non-recycled content. The r-PTA product can then be used for a variety of end-use applications, including for making PET and other polymers. Other end uses of r-PTA include, but are not limited to, as a feedstock for making other chemicals including terephthalate plasticizers like recycled content dioctyl terephthalate (r-dioctyl terephthalate) and recycled content dibutyl terephthalate (r-dibutyl terephthalate), and as an additive for paints and coatings, or as a starting materials for various pharmaceuticals.

As shown in FIG. 4, at least a portion of the r-PTA product may be introduced into a polyethylene terephthalate (PET) production facility, wherein it can be reacted with ethylene glycol to form recycled content PET (r-PET). At least a portion of the r-PTA introduced into the PET production facility can include non-recycled content from the same or a different source.

In one embodiment or in combination with any embodiment mentioned herein, at least a portion of the ethylene glycol (EG) introduced into the PET production facility can include recycled content EG (r-EG) and/or sustainable content EG (s-EG). In some cases, at least a portion of the EG may also include non-recycled content EG, or the EG introduced into the PET production facility may include no recycled content.

When at least a portion of the EG reacted in the PET production facility comprises r-EG, at least a portion of the r-EG may be formed by conversion of recycled content methanol (r-methanol) and/or from the conversion of recycled content ethylene (r-ethylene), as generally shown in FIG. 3b. When the r-EG is formed from r-methanol, it may follow one or more of several chemical pathways. For example, in one embodiment or in combination with any embodiment mentioned herein, r-methanol can be dehydrogenated to form recycled content formaldehyde (r-formaldehyde), which can then be hydrocarboxylated with water and carbon monoxide (or r-carbon monoxide) to form recycled content glycolic acid (r-glycolic acid). The resulting r-glycolic acid can be esterified with methanol (or r-methanol) to provide recycled content methyl glycolate (r-methyl glycolate), which can be hydrogenated (with H2 or r-H2) to form recycled content ethylene glycol (r-EG). The r-EG may then be purified in a separation step to remove byproduct recycled content diethylene glycol (r-DEG) and a portion of the r-EG can be introduced into the PET production facility. Alternatively, the r-formaldehyde formed as described above can be hydroformylated with recycled content syngas (r-syngas) to provide recycled content glycolaldehyde (r-glycolaldehyde), which can then be hydrogenated (with H2 or r-H2) to form recycled content ethylene glycol (r-EG).

The r-methanol used as a starting material for the r-EG can be formed by oxidizing recycled content methane (r-methane), which can originate from one or more of the pyrolysis facility, the steam cracking facility, and/or the refinery (embodiment not shown in FIG. 2). Alternatively, at least a portion of the r-methanol can come from catalytic synthesis of r-syngas. In some cases, at least a portion of the methanol for this or any application described herein can include sustainable content methanol (s-methanol) formed by, for example, processing biomass.

When at least a portion of the r-EG introduced into the PET production facility comes from r-ethylene, at least a portion of the r-ethylene can be oxidized to form recycled content ethylene oxide (r-EO). The r-EO can then be hydrated to provide recycled content ethylene glycol (r-EG), or it can be reacted with carbon dioxide (or recycled content carbon dioxide, r-CO2) and then hydrolyzed to form the r-EG. The r-ethylene used in this reaction pathway can originate from the refinery and/or the steam cracking facility of the chemical recycling facility.

In one embodiment or in combination with any embodiment mentioned herein, the r-EG can come from a solvolysis facility for chemically recycling waste plastic and, in particular, waste PET (not shown). In the solvolysis facility, the waste PET may be broken down into its monomeric components (e.g., dimethyl terephthalate and ethylene glycol) via reaction with heat and catalyst in the presence of a solvent. Examples of solvents include methanol (methanolysis), ethanol (ethanolysis), water (hydrolysis), ethylene glycol (glycolysis), and ammonia (ammonolysis). The resulting monomers, which comprise recycled content, can then be separated and withdrawn as product streams such as recycled content dimethyl terephthalate (r-DMT) and recycled content ethylene glycol (r-EG). In some cases, at least a portion of the r-EG from the solvolysis facility can be introduced into the PET production facility.

As shown in FIG. 3b, at least a portion of the EG introduced into the PET production facility can include sustainable content EG (s-EG) that includes one or more components from a biological source and/or it may include EG that does not include recycled or sustainable content.

As shown in FIGS. 2b and 3, at least one comonomer may also be introduced into the PET production facility to react with the r-PTA and/or EG (or r-EG or s-EG) to provide a recycled content co-polyester (r-co-polyester). The comonomer can comprise a diacid, a diol, a polyacid, or a polyol. In some cases, two or more comonomers may be used and these comonomers can be of the same (e.g., both diols) or different (e.g., a diol and a diacid) types. The comonomer or comonomers may or may not include recycled content. When present, the comonomer or comonomers can make up at least 5, at least 10, at least 15 and/or not more than 45, not more than 40, not more than 35, not more than 30, not more than 25, not more than 20, not more than 15, or not more than 10 mole percent of the co-PET acid or diol component. When the comonomer is a diacid or polyacid, the percentages are determined based on the total acid component as 100 percent, and when the comonomer is a diol or polyol, the percentages are determined based on the total diol component as 100 percent.

Examples of suitable diacid comonomers (or r-comonomers) include, but are not limited to, isophthalic acid, 1,4-cyclohexanedicarboxylic acid, phthalic acid, naphthalene-2,6-dicarboxylic acid, cyclohexanediacetic acid, diphenyl-4,4′-dicarboxylic acid, diphenyl-3,4′-dicarboxylic acid, 2,2-dimethyl-1,3-propandiol, dicarboxylic acid, succinic acid, glutaric acid, adipic acid, azelaic acid, sebacic acid, and mixtures thereof. Suitable diol comonomers can include, for example, 1,4-cyclohexanedimethanol (1,4-CHDM), 2,2,4,4-trimethane-cyclo-1,3-butandiol (2,2,4,4-TMCD), neopentyl glycol (NPG), and diethylene glycol (DEG), isosorbide, 1,4-butanediol, and 1,3-propane diol. In one embodiment or in combination with any embodiment mentioned herein, the comonomer may comprise isophthalic acid (PIA) or recycled content isophthalic acid (r-PIA). In some cases, at least a portion of the r-PIA can be formed by oxidizing recycled content metaxylene (r-metaxylene) in an isophthalic acid (PIA) production facility as shown in FIG. 2. At least a portion of the r-metaxylene can originate from the same, or a different, aromatics complex than the r-paraxylene used to form the r-PTA.

In one embodiment or in combination with one or more embodiments mentioned herein, the r-PET may not include significant amounts comonomer, such that the r-PET is considered a PET homopolymer. In such cases, the total amount of comonomers (based on the total acid and total diol components) present in the r-PET can be not more than 6, not more than 5, not more than 4, not more than 3, not more than 2, or not more than 1 mole percent, based on the total acid and/or diol component.

As shown in FIG. 4, the r-PTA (and optionally PTA) may be introduced with ethylene glycol (r-EG, s-EG, and/or EG) into an esterification zone/step, wherein the acid and glycols can be polymerized to form r-PET oligomers. When present, at least one diacid and/or diol comonomer (or r-comonomer) may also be added and reacted to form r-co-PET oligomers. The esterification can be carried out at or below atmospheric pressure and in the presence of a catalyst. The r-oligomer withdrawn from the esterification zone can be introduced into a downstream polymerization zone/step, as shown in FIG. 4, wherein further reaction can occur to increase the molecular weight of the resin. Once the polymerization step is complete, the molten r-PET can be sent to a pelletization and crystallization zone, wherein the r-PET pellets can be formed and crystallized, as well as dried and further treated to remove residual impurities (e.g., acetaldehyde). In some cases, the r-PET pellets can be solid state polymerized to achieve even higher molecular weights (measured as inherent viscosity, I.V.), while, in other cases, no solid-state polymerization is used. The final r-PET pellets can be withdrawn from the PET production facility and routed to one or more downstream facilities for further processing and/or use.

In one embodiment or in combination with any embodiment mentioned herein, at least a portion of the r-PET pellets can be used to form at least one r-PET article. Such formation typically includes melting the r-PET to form an r-PET melt and then extruding or otherwise shaping the r-PET to form a recycled content PET article (r-PET article). Examples of r-PET articles can include, but are not limited to, bottles, containers, preforms, films, sheets, and other similar articles, which can be used alone or further processed to form an article for the desired end use.

### DEFINITIONS

It should be understood that the following is not intended to be an exclusive list of defined terms. Other definitions may be provided in the foregoing description, such as, for example, when accompanying the use of a defined term in context.

As used here, the term “light gas” refers to a hydrocarbon-containing stream comprising at least 50 weight percent of C4 and lighter hydrocarbon components. Light hydrocarbon gas may include other components such as nitrogen, carbon dioxide, carbon monoxide, and hydrogen, but these are typically present in amount of less than 20, less than 15, less than 10, or less than 5 weight percent, based on the total weight of the stream.

As used herein, the terms “median boiling point” or “T50” refers to the median boiling point of a process stream (i.e., the temperature value where 50 weight percent of the stream composition boils above the temperature value and 50 weight percent of the stream composition boils below the temperature value).

As used herein, the term “boiling point range” or “cut point” refers to the range of temperatures over which a specified petroleum fraction boils. The lower value in a boiling point range is the initial boiling point (IBP) temperature for that specified fraction and the higher value is the end point (EP) temperature for that specified fraction.

As used herein, the term “naphtha” refers to a physical mixture of hydrocarbon components separated in at least one distillation column of a refining facility that has a boiling point range between 90 to 380° F.

As used herein, the term “light naphtha” refers to a specific portion of a naphtha cut in a refinery having a boiling point range between 90 and 190° F.

As used herein, the term “heavy naphtha” refers to a specific portion of a naphtha cut in a refinery having a boiling point range between 190 and 380° F.

As used herein, the terms “distillate” and “kerosine” refer to a physical mixture of hydrocarbon components separated in at least one distillation column of a refining facility that has a boiling point range greater than 380 to 520° F.

As used herein, the term “hydrocracker distillate” refers to a distillate cut removed from a hydrocracker unit.

As used herein, the term “gas oil” refers to a physical mixture of hydrocarbon components separated in at least one distillation column of a refining facility that has a boiling point range greater than 520 to 1050° F.

As used herein, the term “atmospheric gas oil” refers to a gas oil produced by the atmospheric distillation unit.

As used herein, the term “light gas oil” or “LGO” refers to a specific portion of gas oil cut in a refinery having a boiling point range between greater than 520 and 610° F.

As used herein, “light vacuum gas oil” or “LVGO” refers to a light gas oil produced by the vacuum distillation unit.

As used herein, “light vacuum gas oil” or “LCGO” refers to a light gas oil produced by the coker unit.

As used herein, the term “heavy gas oil” or “HGO” refers to a specific portion of a gas oil cut in a refinery having a boiling point range between greater than 610 and 800° F.

As used herein, “heavy vacuum gas oil” or “HVGO” refers to a heavy gas oil produced by the vacuum distillation unit.

As used herein, “heavy coker gas oil” or “HCGO” refers to a heavy gas oil produced by the coker unit.

As used herein, the term “vacuum gas oil” or “VGO” refers to a specific portion of a gas oil cut in a refinery having a boiling point range between greater than 800 and 1050° F. Vacuum gas oil is separated from the initial crude oil using a vacuum distillation column operated at a pressure below atmospheric pressure.

As used herein, the term “residue” or “resid” refers to the heaviest cut from a distillation column in a refinery and having a boiling point range between greater than 1050° F.

As used herein, the term “vacuum resid” refers to a resid product from the vacuum distillation column.

As used herein, the term “atmospheric resid” refers to a resid product from the atmospheric distillation column.

As used herein, the term “gas plant” refers to equipment, including one or more distillation columns as well as ancillary equipment as well as pumps, compressors, valves, etc. in a refinery for processing a hydrocarbon feed stream comprising predominantly C6 and lighter components to provide one or more purified streams of C1 to C6 alkanes and/or olefins.

As used herein, the term “saturated gas plant” refers to a gas plant in a refinery for processing a hydrocarbon feed stream comprising predominantly saturated hydrocarbons (alkanes). The feed stream to a saturated gas plant includes less than 5 weight percent of olefins, based on the total feed to the plant. The feed to a saturated gas plant in a refinery may come, directly or indirectly, from the crude distillation unit or vacuum distillation unit and may undergo little or no cracking.

As used herein, the term “unsaturated gas plant” refers to a gas plant in a refinery for processing a hydrocarbon feed stream comprising saturated hydrocarbons (alkanes) and unsaturated hydrocarbons (olefins). The feed stream to an unsaturated gas plant includes at least 5 weight percent of olefins, based on the total feed to the plant. The feed to a saturated gas plant in a refinery may come indirectly from the crude unit or vacuum distillation unit and may undergo one or more cracking steps prior to entering the gas plant.

As used herein, the term “gas oil cracker” refers to a cracking unit for processing a feed stream comprising predominantly gas oil and heavier components. Although a gas oil cracker can process lighter components, such as distillate and naphtha, at least 50 weight percent of the total feed to a gas oil cracker includes gas oil and heavier components. Gas oil crackers may be operated at temperatures of at least 350° F., at least 400° F., at least 450° F., at least 500° F., at least 550° F., or at least 600° F. and/or not more than 1200° F., not more than 1150° C., not more than 1100° F., not more than 1050° F., not more than 1000° F., not more than 900° F., or not more than 800° F. Gas oil crackers may be operated at or near atmospheric pressure (e.g., at a pressure of less than 5 psig, less than 2 psig, or 1 psig) or may be operated at elevated pressure (e.g., at a pressure of at least 5 psig, at least 10 psig, at least 25 psig, at least 50 psig, at least 100 psig, at least 250 psig, at least 500 psig, or at least 750 psig.)

Additionally, the cracking in gas oil crackers may be carried with or without a catalyst, and it may or may not be conducted in the presence of hydrogen and/or steam.

As used herein, the term “fluidized catalytic cracker” or “FCC” refers to a set of equipment, including a reactor, a regenerator, a main fractionator, as well as ancillary equipment such as pipes, valves, compressors, and pumps, for reducing the molecular weight of a heavy hydrocarbon stream via catalytic cracking in a fluidized catalyst bed.

As used herein, the terms “reformer” or “catalytic reformer” refer to a process or facility in which a feedstock comprising predominantly C6-C10 alkanes is converted to a reformate comprising branched hydrocarbons and/or cyclic hydrocarbons in the presence of a catalyst.

As used herein, the term “reformate” refers to a liquid product stream produced by a catalytic reformer process.

As used herein, the term “hydroprocessing” refers to chemical processing of a hydrocarbon stream with or in the presence of hydrogen. Hydroprocessing is typically a catalytic process and includes hydrocracking and hydrotreating.

As used herein, the term “hydrocracking” refers a type of hydroprocessing where the hydrocarbon molecules are cracked (i.e., undergo a reduction in molecular weight).

As used herein, the term “hydrotreating” refers to a type of hydroprocessing that does not crack the hydrocarbon molecules, but instead removes oxygen, sulfur, and other heteroatoms by hydrogenolysis or to saturate unsaturated bonds by hydrogenation. It may or may not be carried out in the presence of a catalyst.

As used herein, the term “distillation” refers to separation of a mixture of components by boiling point difference.

As used herein, the term “atmospheric distillation” refers to distillation performed at a pressure at or near atmospheric, usually to separate crude oil and/or other streams into specified fractions for further processing.

As used herein, the term “vacuum distillation” refers to distillation performed at a pressure below atmospheric and, usually, at a pressure of less than 100 mm Hg at the top of the column.

As used herein, the term “coking” refers to thermal cracking of heavy hydrocarbons (usually atmospheric or vacuum column bottoms) performed to recover light, more valuable products such as naphtha, distillate, gas oil, and light gas.

As used herein, the term “aromatics complex” refers to a process or facility in which a mixed hydrocarbon feedstock, such as a reformate, is converted into one or more benzene, toluene, and/or xylene (BTX) product streams, such as a paraxylene product stream. The aromatics complex may comprise one or more processing steps, in which one or more components of the reformate are subjected to at least one of a separation step, a transalkylating step, a toluene disproportionation step, and/or an isomerization step. The separation step can comprise one or more of an extraction step, a distillation step, a crystallization step, and/or an adsorption step.

As used herein, the term “raffinate” refers to the aromatics-depleted stream removed from the initial separation step in the aromatics complex. Although most commonly used to refer to a stream withdrawn from an extraction step, the term “raffinate” as used with respect to the aromatics complex can also refer to a stream withdrawn from another type of separation, including, but not limited to, distillation or extractive distillation.

As used herein, the terms “pyrolysis oil” or “pyoil” refers to a composition obtained from pyrolysis that is liquid at 25° C. and 1 atm, absolute.

As used herein, the terms “pyrolysis gas” and “pygas” refer to a composition obtained from pyrolysis that is gaseous at 25° C. and 1 atm, absolute.

As used herein, the term “pyrolysis” refers to thermal decomposition of one or more organic materials at elevated temperatures in an inert (i.e., substantially oxygen free) atmosphere.

As used herein, the term “pyrolysis vapor” refers to the overhead or vapor-phase stream withdrawn from the separator in a pyrolysis facility used to remove r-pyrolysis residue from the r-pyrolysis effluent.

As used herein, the term “pyrolysis effluent” refers to the outlet stream withdrawn from the pyrolysis reactor in a pyrolysis facility.

As used herein, the term “r-pyrolysis residue” refers to a composition obtained from waste plastic pyrolysis that comprises predominantly pyrolysis char and pyrolysis heavy waxes.

As used herein, the term “pyrolysis char” refers to a carbon-containing composition obtained from pyrolysis that is solid at 200° C. and 1 atm, absolute.

As used herein, the term “pyrolysis heavy waxes” refers to C20+hydrocarbons obtained from pyrolysis that are not pyrolysis char, pyrolysis gas, or pyrolysis oil.

As used herein, the term “pyrolysis gasoline” refers to a hydrocarbon stream of predominantly C5 and heavier components removed from a quench section of a steam cracking facility. Typically, pyrolysis gasoline includes at least 10 weight percent of C6 to C9 aromatics.

As used herein, the term “lighter” refers to a hydrocarbon component or fraction having a lower boiling point than another hydrocarbon component or fraction.

As used herein, the term “heavier” refers to a hydrocarbon component or fraction having a higher boiling point than another hydrocarbon component or fraction.

As used herein, the term “upstream” refers to an item of facility that is positioned prior to another item or facility in a given process flow and may include intervening items and/or facilities.

As used herein, the term “downstream” refers to an item or facility that is positioned after another item or facility in a given process flow and may include intervening items and/or facilities.

As used herein, the term “alkane” refers to a saturated hydrocarbon including no carbon-carbon double bonds.

As used herein, the term “olefin” refers to an at least partially unsaturated hydrocarbon including at least one carbon-carbon double bond.

As used herein, the terms “Cx” or “Cx hydrocarbon” or “Cx component” refers to a hydrocarbon compound including “x” total carbons per molecule, and encompasses all olefins, paraffins, aromatics, heterocyclic, and isomers having that number of carbon atoms. For example, each of normal, iso, and tert-butane and butene and butadiene molecules would fall under the general description “C4” or “C4 components.”

As used herein, the terms “r-paraxylene” or “r-pX” refer to being or comprising a paraxylene product that is directly and/or indirectly derived from waste plastic.

As used herein, the term “cracking” refers to breaking down complex organic molecules into simpler molecules by the breaking of carbon-carbon bonds.

As used herein, the term “steam cracking” refers to thermal cracking of hydrocarbons in the presence of steam, usually performed in a furnace of the steam cracking facility.

As used herein, the term “co-located” refers to the characteristic of at least two objects being situated on a common physical site, and/or within five miles of each other, measured as a straight-line distance between two designated points.

As used herein, the term “commercial scale facility” refers to a facility having an average annual feed rate of at least 500 pounds per hour, averaged over one year.

As used herein, the terms “crude” and “crude oil” refer to a mixture of hydrocarbons that exists in liquid phase and is derived from natural underground reservoirs.

As used herein, the terms “recycle content” and “r-content” refer to being or comprising a composition that is directly and/or indirectly derived from waste plastic.

As used herein, the term “predominantly” means more than 50 percent by weight. For example, a predominantly propane stream, composition, feedstock, or product is a stream, composition, feedstock, or product that contains more than 50 weight percent propane.

As used herein, the term “waste material” refers to used, scrap, and/or discarded material.

As used herein, the terms “waste plastic” and “plastic waste” refer to used, scrap, and/or discarded plastic materials.

As used herein, the terms “mixed plastic waste” and “MPW” refer to a mixture of at least two types of waste plastics including, but not limited to the following plastic types: polyethylene terephthalate (PET), one or more polyolefins (PO), and polyvinylchloride (PVC).

As used herein, the term “fluid communication” refers to the direct or indirect fluid connection between two or more processing, storage, or transportation facilities or zones.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and propylene) that are useful by themselves and/or are useful as feedstocks to another chemical production process(es).

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the term “cracking” refers to breaking down complex organic molecules into simpler molecules by the breaking of carbon-carbon bonds.

As used herein, the terms “including,” “include,” and “included” have the same open-ended meaning as “comprising,” “comprises,” and “comprise” provided above.

As used herein, the term “predominantly” means more than 50 percent by weight. For example, a predominantly propane stream, composition, feedstock, or product is a stream, composition, feedstock, or product that contains more than 50 weight percent propane.

As used herein, the term “hydrocarbon” refers to an organic chemical compound that includes only carbon and hydrogen atoms.

As used herein, the term “organic chemical compound,” refers to a chemical compound that includes carbon and hydrogen atoms, but also includes oxygen and/or nitrogen atoms.

As used herein, the term “diol” refers to an alcohol having two or more hydroxyl groups.

As used herein, the term “diacid” refers to an acid, and particularly a dicarboxylic acid, having two or more acid, or carboxylic acid, groups.

As used herein, the term “diester” refer to an ester compound including two or more ester groups.

As used herein, the term “chemical pathway” refers to the chemical processing step or steps (e.g., chemical reactions, physical separations, etc.) between an input material and a product, where the input material is used to make the product.

As used herein, the terms “credit-based recycled content,” “non-physical recycled content,” and “indirect recycled content” all refer to matter that is not physically traceable back to a waste material, but to which a recycled content credit has been attributed.

As used herein, the term “directly derived” refers to having at least one physical component originating from waste material.

As used herein, the term “indirectly derived” refers to having an applied recycled content (i) that is attributable to waste material, but (ii) that is not based on having a physical component originating from waste material.

As used herein, the term “located remotely” refers to a distance of at least 0.1, 0.5, 1, 5, 10, 50, 100, 500, or 1000 miles between two facilities, sites, or reactors.

As used herein, the term “mass balance” refers to a method of tracing recycled content based on the mass of the recycled content in a product.

As used herein, the terms “physical recycled content” and “direct recycled content” both refer to matter physically present in a product and that is physically traceable back to a waste material.

As used herein, the term “recycled content” refers to being or comprising a composition that is directly and/or indirectly derived from recycle waste material. Recycled content is used generically to refer to both physical recycled content and credit-based recycled content. Recycled content is also used as an adjective to describe a product having physical recycled content and/or credit-based recycled content.

As used herein, the term “recycled content credit” refers to a non-physical measure of recycled content obtained from a mass of waste plastic that can be directly or indirectly (i.e., via a digital inventory) attributed to a product second material.

As used herein, the term “total recycled content” refers to the cumulative amount of physical recycled content and credit-based recycled content from all sources.

As used herein, the term “waste material” refers to used, scrap, and/or discarded material.

As used herein, the terms “waste plastic” and “plastic waste” refer to used, scrap, and/or discarded plastic materials, including post-industrial or pre-consumer waste plastic and post-consumer waste plastic.

As used herein, the term “hydroprocessing unit” refers to a set of equipment, including reaction vessels, a drier, and a main fractionator, as well as ancillary equipment such as pipes, valves, compressors, and pumps, for chemically processing a hydrocarbon stream in the presence of hydrogen. Specific examples of hydroprocessing units include a hydrocracker (or hydrocracking unit) configured to carry out a hydrocracking process and a hydrotreater (or hydrotreating unit) configured to carry out a hydrotreating process.

As used herein, the term “coker” or “coking unit” refers to a set of equipment, including reaction vessels, a drier, and a main fractionator, as well as ancillary equipment such as pipes, valves, compressors, and pumps, for reducing the molecular weight of a heavy hydrocarbon stream via thermal cracking or coking.

As used herein, the terms “steam cracking facility” or “steam cracker” refer to all of the equipment needed to carry out the processing steps for thermally cracking a hydrocarbon feed stream in the presence of steam to form one or more cracked hydrocarbon products. Examples include, but are not limited to, olefins such as ethylene and propylene. The facility may include, for example, a steam cracking furnace, cooling equipment, compression equipment, separation equipment, as well as the pipes, valves, tanks, pumps, etc. needed to carry out the processing steps.

As used herein, the terms “refinery,” “refining facility,” and “petroleum refinery,” refer to all of the equipment needed to carry out the processing steps for separating and converting petroleum crude oil into multiple hydrocarbon fractions, one or more of which can be used as a fuel source, lube oil, bitumen, coke, and as an intermediate for other chemical products.” The facility may include, for example, separation equipment, thermal or catalytic cracking equipment, chemical reactors, and product blending equipment, as well as the pipes, valves, tanks, pumps, etc. needed to carry out the processing steps.

As used herein, the term “pyrolysis facility,” refers to all of the equipment needed to carry out the processing steps for pyrolyzing a hydrocarbon-containing feed stream, which can include or be waste plastic. The facility may include, for example, reactors, cooling equipment, and separation equipment, as well as the pipes, valves, tanks, pumps, etc. needed to carry out the processing steps.

As used herein, the term “terephthalic acid production facility,” or “TPA production facility,” refers to all of the equipment needed to carry out the processing steps for forming terephthalic acid from paraxylene. The facility may include, for example, reactors, separators, cooling equipment, separation equipment such as filters or crystallizers, as well as the pipes, valves, tanks, pumps, etc. needed to carry out the processing steps.

As used herein, the term “polyethylene terephthalate production facility,” or “PET production facility,” refers to all of the equipment needed to carry out the processing steps for forming polyethylene terephthalate (PET) from a terephthalate, ethylene glycol, and, optionally, one or more additional monomers. The facility may include, for example, polymerization reactors, cooling equipment, and equipment to recover solidified and/or pelletized PET, as well as the pipes, valves, tanks, pumps, etc. needed to carry out the processing steps.

As used herein the term “chemical processing facility,” refers to all of the equipment needed to carry out the processing steps for one or more chemical processes for converting a starting material to a final chemical product. The facility may include, for example, separation or treatment equipment, reaction equipment, and equipment to recover a final product, as well as the pipes, valves, tanks, pumps, etc. needed to carry out the processing steps.

### CLAIMS NOT LIMITED TO DISCLOSED EMBODIMENTS

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

What is claimed is:

1. A process for producing a recycled content organic chemical compound (r-chemical compound), said process comprising:
(a) oxidizing a recycled content paraxylene (r-paraxylene) stream in an oxidation zone of a terephthalic acid (TPA) production facility to provide a recycled content crude terephthalic acid (r-CTA) slurry, wherein the r-paraxylene stream includes recycled content derived from waste plastic;
(b) treating at least a portion of the r-CTA slurry to provide a liquid stream comprising recycled content purified terephthalic acid (r-PTA); and
(c) crystallizing at least a portion of the liquid stream comprising r-PTA to provide a recycled content purified terephthalic acid (r-PTA) product.

2. The process of claim 1, wherein the r-CTA slurry comprises a first solvent comprising acetic acid.

3. The process of claim 2, further comprising prior to the treating of step (b) removing at least a portion of the first solvent from the r-CTA slurry to provide r-CTA and combining the r-CTA with a second solvent to provide a second r-CTA slurry, wherein the treating of step (b) comprises treating the second r-CTA slurry to provide the liquid stream comprising r-PTA.

4. The process of claim 3, wherein the second solvent comprises predominantly acetic acid and wherein the treating of step (b) includes further oxidation of the second r-CTA slurry.

5. The process of claim 3, wherein the second solvent comprises predominantly water and wherein the treating of step (b) includes hydrogenation of the second r-CTA slurry.

6. The process of claim 1, wherein the r-paraxylene stream comprises non recycled content.

7. The process of claim 1, further comprising, prior to the oxidizing of step (a), converting waste plastic to a recycled content aromatics (r-aromatics) stream and treating at least a portion of the r-aromatics stream in an aromatics complex to provide the r-paraxylene stream.

8. The process of claim 1, further comprising reacting at least a portion of the r-PTA with ethylene glycol in a PET production facility to provide a recycled content polyethylene terephthalate (r-PET) and wherein the EG comprises recycled content ethylene glycol (r-EG).

9. A process for producing a recycled content organic chemical compound (r-organic chemical compound), said process comprising:
(a) converting waste plastic in at least one conversion facility to form a recycled content aromatics (r-aromatics) stream comprising recycled content paraxylene (r-px);
(b) separating at least a portion of the r-aromatics stream in an aromatics complex to provide a recycled content paraxylene (r-paraxylene) stream;
(c) oxidizing at least a portion of the r-paraxylene stream in an oxidation zone of a terephthalic acid production facility to provide a recycled content crude terephthalic acid (r-CTA) slurry; and
(d) processing at least a portion of the r-CTA slurry to provide a stream of recycled content purified terephthalic acid (r-PTA).

10. The process of claim 9, wherein the conversion facility comprises a steam cracking facility for processing at least one recycled content hydrocarbon (r-HC) stream and wherein the r-aromatics stream produced in the steam cracking facility comprises recycled content pyrolysis gasoline (r-pyrolysis gasoline), wherein the r-HC stream comprises recycled content pyrolysis oil (r-pyoil) and/or recycled content pyrolysis gas (r-pygas) from a pyrolysis facility for pyrolyzing waste plastic and/or recycled content naphtha (r-naphtha) and/or recycled content light gas (r-light gas) from a refinery for processing at least one stream of r-pyoil and/or r-pygas from a pyrolysis facility for pyrolyzing waste plastic.

11. The process of claim 9, wherein the conversion facility comprises a refinery for processing at least one recycled content hydrocarbon (r-HC) stream and wherein the r-aromatics stream produced in the steam cracking facility comprises recycled content reformate (r-reformate), wherein the r-HC stream comprises at least one of recycled content pyrolysis oil (r-pyoil) from a pyrolysis facility for pyrolyzing waste plastic, recycled content naphtha (r-naphtha) from an atmospheric distillation unit (ADU) and r-naphtha from a gas oil cracker in the refinery.

12. The process of claim 9, wherein the conversion facility comprises a methanol-to-aromatics facility configured to process recycled content methanol (r-methanol) to form the r-aromatics stream, wherein the r-methanol comes from a methanol synthesis facility for converting recycled content syngas (r-syngas) to r-methanol, and wherein the r-syngas comes from a molecular reforming facility for processing a feed stream comprising waste plastic or one or more streams comprising recycled content derived from waste plastic.

13. The process of claim 9, wherein the conversion facility comprises a cracking facility for thermally cracking waste polyethylene terephthalate (PET) to form a recycled content cracked PET (r-cracked PET) stream and separating at least a portion of the r-cracked PET stream to provide the r-aromatics stream.

14. The process of claim 9, wherein the separating of step (b) includes an initial separation step for separating the r-aromatics stream into a recycled content benzene, toluene, and xylenes (r-BTX) stream and a recycled content raffinate (r-raffinate) stream and further comprising processing at least a portion of the r-raffinate stream in another facility to provide another r-aromatics stream and introducing at least a portion of another r-aromatics stream into the aromatics complex.

15. The process of claim 14, further comprising subjecting at least a portion of the r-BTX stream to one or more additional processing steps to provide the r-paraxylene stream and wherein the r-paraxylene stream comprises at least 85 weight percent of paraxylene.

16. The process of claim 9, wherein the processing of step (d) includes treating the r-CTA slurry to provide a treated r-CTA slurry and crystallizing at least a portion of the treated r-CTA slurry to provide the r-PTA, and wherein the r-PTA includes at least 97 weight percent terephthalic acid.

17. The process of claim 9, wherein at least two of the conversion facility, the aromatics complex, and the TPA production facility are commercial scale facilities and/or are co-located.

18. A process for producing a recycled content organic chemical compound (r-organic chemical compound), said process comprising:
(a) forming recycled content purified terephthalic acid (r-PTA) from a recycled content paraxylene (r-paraxylene) stream in a terephthalic acid (TPA) production facility, wherein the r-paraxylene stream includes recycled content derived from waste plastic; and
(b) reacting at least a portion of the r-PTA with ethylene glycol (EG) to form recycled content polyethylene terephthalate (r-PET).

19. The process of claim 18, wherein at least a portion of the r-paraxylene stream is obtained by processing a recycled content aromatics (r-aromatics) stream in an aromatics complex and wherein the r-aromatics stream comes from at least one of the following steps (i) through (iv) below:
(i) thermally cracking waste polyethylene terephthalate (PET) to form a recycled content cracked PET (r-cracked PET) stream and separating at least a portion of the r-cracked PET stream to provide the r-aromatics stream;
(ii) reforming at least one recycled content hydrocarbon (r-HC) stream in a refinery to provide at least a portion of the r-aromatics stream, wherein the r-HC stream comprises at least one of r-pyoil, r-naphtha, and r-cracked naphtha from a pyrolysis facility and/or a refinery;
(iii) processing at least one recycled content hydrocarbon (r-HC) stream in a steam cracking facility to provide at least a portion of the r-aromatics stream, wherein the r-HC stream comprises at least one of r-pyoil, r-pygas, r-naphtha, r-cracked naphtha, and r-light gas from a pyrolysis facility and/or a refinery; and
(iv) processing at least one recycled content synthesis gas (r-synags) and/or at least one recycled content methanol (r-methanol) stream to provide at least a portion of the r-aromatics in a methanol-to-aromatics (MTA) facility, wherein the r-syngas and/or r-methanol comprise recycled content from waste plastic.

20. The process of claim 18, wherein the forming of step (a) includes oxidizing at least a portion of the r-paraxylene stream in an oxidation zone to form a recycled content crude terephthalic acid (r-CTA), wherein the oxidizing is carried out in a presence of a first solvent comprising acetic acid to provide an r-CTA slurry.
